(19) United States
(12) Patent Application Publication
(10) Pub. No.: US 2024/0139235 A1
(43) Pub. Date: May 2, 2024

(54) IRON(III) FORMULATIONS FOR THE TREATMENT OF GASTROINTESTINAL INFLAMMATION

(71) Applicant: PHARMANUTRA S.P.A., Pisa(PI) (IT)

(72) Inventors: Andrea LACORTE, Pisa (PI) (IT); Germano TARANTINO, Pisa (PI) (IT); Elisa BRILLI, Pisa (PI) (IT)

(21) Appl. No.: 18/279,039

(22) PCT Filed: Mar. 14, 2022
(86) PCT No.: PCT/IB2022/052257

§ 371 Date: Aug. 25, 2023

(30) Foreign Application Priority Data
Mar. 12, 2021 (IT) 102021000005981

(51) Int. Cl. A61K 33/26; A61K 33/42; A61K 47/24; A61K 47/26; A61K 47/36; A61P 1/04

(52) U.S. Cl. CPC A61K 33/26; A61K 33/42; A61K 47/24; A61K 47/26; A61K 47/36; A61P 1/04

(57) ABSTRACT

The present invention relates to an iron-based formulation and compositions thereof for use in a method for the treatment of a gastrointestinal inflammation, in a therapeutic or non-therapeutic treatment of an iron deficiency by administering said formulation to a subject, wherein said iron formulation is capable of reducing (or maintaining constant) the amount of pathogenic bacteria present in the gut microbiota of the treated subject and, preferably, also capable of increasing the variety of the gut microbiota of the treated subject.

The present invention relates to an iron-based formulation, and compositions comprising it, for use in a method for the treatment of a gastrointestinal infection in subjects subjected to a treatment of an iron deficiency. Said treatment method allows to reduce the pathological side effects, or the worsening thereof, such as gastrointestinal disorders, which arise in a subject subjected to a therapeutic or non-therapeutic treatment of an iron deficiency by administering said iron formulation. In particular, said iron formulation, and the compositions comprising it, are capable of reducing the amount of pathogenic bacteria (or maintaining it constant or combating the growth thereof), preferably belonging to the phylum Proteobacteria, present in the gut microbiota of the treated subject. Furthermore, said iron formulation, and the compositions comprising it, are advantageously capable of increasing the variety of the gut microbiota of the treated subject by shifting the balance toward the species beneficial to the organism and, therefore, they are capable of reducing the side effects that arise.

It is known that following an administration of an iron-based supplement, selected from those currently available on the market, such as for example those based on Fe (II), a prevalence (or increase or rise) of pathogenic bacteria (e.g. of phylum Proteobacteria) may occur in the (gastro)intestinal microbiota of the treated subject who takes said iron-based supplement.

Gut dysbiosis is a condition of microbial imbalance caused by an overgrowth of so-called “bad” bacteria inside the gut, which cause the irritation thereof. The microbiome, the set of symbiotic bacteria that coexist in our organism, particularly in the gut, is closely related to the immune system, thus playing a very important role in regulating immune tolerance. When the balance of the gut microbiome changes, it is called gut dysbiosis, a disorder that can also be caused by poor nutrition and it is related to chronic inflammatory diseases, obesity, tumours and colitis.

Said increase in gastrointestinal pathogens, following an iron supplementation, has been particularly evident in geographical areas or in subjects in whom the presence of gastrointestinal pathogens is endemic (such as for example in underdeveloped countries and/or in undernourished subjects). The prevalence of gastrointestinal pathogenic bacteria increases the risk of gastrointestinal infections and of diseases and/or disorders associated with said intestinal infections, such as for example intestinal inflammations, gastrointestinal ulcers, chronic gastritis, gastric atrophy, intestinal diverticula, diarrhoea, vomiting, nausea and gastrointestinal pain.

In addition, said gastrointestinal infections or disorders reduce the absorption of iron administered through said iron-based supplements.

Furthermore, subjects with iron deficiency, even mild, generally exhibit an imbalance of the gut microbiota and, particularly, a decrease in the diversity of the gastrointestinal microbiota with reduction of the beneficial bacteria known as producers of short chain fatty acids. This means that subjects with iron deficiencies (not necessarily anaemic) can contract gastrointestinal infections more easily with respect to subjects not affected by iron deficiency.

Document WO2019/025922 relates to a composition comprising an iron (III) salt, a phospholipid and a sucrester for the treatment of an iron deficiency.

The article by Abbati G. et al. XP036744615 relates to the use of an iron-based composition referred to as Sucrosomial® Iron for the treatment of gastrointestinal disorders.

The article by Susana Gomez-Ramirez et al. XP055665686 relates to the use of an iron-based composition referred to as Sucrosomial® Iron for the treatment of gastrointestinal disorders.

The article by Angela Fabiano et al. XP055665690 generally relates to a study on gastrointestinal absorption of an iron-based composition referred to as Sucrosomial® Iron, but it does not disclose the treatment of gastrointestinal inflammations.

The article by Angela Fabiano et al. XP085262682 generally relates to a study on absorption in in-vivo and in-vitro models, but it does not disclose the treatment of gastrointestinal inflammations.

The article by Das Nupur et al. XP055855831 generally relates to the gut microbiota and the modulation thereof.

To date, there are many iron-based supplements available on the market today which can induce gastrointestinal side effects such as diarrhoea, nausea, vomiting and abdominal pain.

Furthermore, excess free iron in the intestinal lumen can potentially lead to the proliferation of pathogenic bacteria in the gut to the detriment of the basic gut microbiome, causing dysbiosis and increasing the risk of gastrointestinal infections.

In the light of the above, the technical problem addressed and solved by the present invention lies in providing an iron supplementation composition which, besides supplying iron, prevents or reduces gastrointestinal infections or disorders, such as pathological side effects associated with the administration of common iron-based supplements.

In other words, the technical problem addressed and solved by the present invention lies in providing an iron supplementation composition which, besides supplying iron, does not cause and/or prevents an increase in pathogenic bacteria (e.g. phylum Proteobacteria) in the (gastro)intestinal microbiota, and/or which is active in restoring balance and adequate diversity of the (gastro)intestinal microbiota and which, as a result, prevents or reduces gastrointestinal infections or disorders, such as pathological side effects associated with common iron-based supplements.

Furthermore, there arises the need to have an iron supplementation composition that does not exacerbate the endemic conditions of the presence of pathogenic bacteria (e.g. of phylum Proteobacteria) in the gastrointestinal microbiota, like in subjects with diseases that lead to an impairment of the gut microbiota (IBD, celiac disease, etc.) or in subjects with inadequate nutritional intake or living in poor hygiene conditions.

Restoring an adequate diversity of the (gastro)intestinal microbiota mainly, but not only, consists of reducing or avoiding the increase of pathogenic bacteria (phylum Proteobacteria) and increasing bacteria capable of producing short-chain fatty acids.

Following an intense research and development activity, the Applicant addresses and solves the technical problems reported above by providing, for the (therapeutic or non-therapeutic) treatment of an iron deficiency, an iron formulation in solid form comprising an iron (III) salt, a phospholipid and a sucrester, as described and claimed (in short, iron formulation according to the present invention or Sucrosomal® iron).

The iron formulations according to the present invention (Sucrosomial® Iron) have shown to increase the gut microbiota diversity in a greater way than a comparative composition for example comprising ferrous sulfate. In particular, the iron formulations according to the present invention have shown to have a positive effect on the reduction of pathogenic bacteria belonging to the phylum Proteobacteria and on the increase of bacteria capable of producing short chain fatty acids.

These and other objects which will be apparent from the detailed description that follows, are achieved by the iron formulation in solid form of the present invention, and by the relative composition, thanks to the technical characteristics reported in the present description and claimed in the attached claims.

### DESCRIPTION OF THE FIGURES

FIGS. 1A-D are a representation of body weight (A), hepatic iron concentration (B), serum iron (C) and transferrin saturation (D) in the 3 groups of mice (ID, FS and IS). All data represent mean±SEM with 10 mice per group (*** P<0.005).

FIGS. 2A-D are a representation of the number of red blood cells (A), haemoglobin (B), haematocrit (C) and mean corpuscular volume (D). All data represent mean±SEM with 10 mice per group (* P<0.05, *** P<0.005).

FIG. 3 is a measurement representation of the microbial composition through beta diversity on faecal samples collected from each mouse after two weeks with a deficient diet (A or baseline) and after two weeks with diets containing iron under analysis (B or FS_B, ID_B, SI_B).

FIGS. 4A-B are a measurement representation of microbial diversity in a single sample: FIG. 4A shows the evenness, richness and Shannon index for each group of animals; FIG. 4B shows changes in Shannon Index and richness in each mouse after two weeks with a specific iron diet (B) with respect to the baseline (A).

FIGS. 5A and 5B are a representation of differences in the microbial composition at the level of Phylum (Proteobacteria and Bacteroidota) in mice fed with Sucrosomial® iron (IS) or ferrous sulfate (FS): directional changes which occur in the microbiome of each mouse after two weeks with the indicated specific diet (B) with respect to the baseline (A). FIGS. 6A-6G are a representation of differences in the microbial composition at the level of Amplicon Single Variant (Lachnospiraceae spp (asv33; asv24; asv72), Rikenellaceae RC9 (asv30; asv151), Faecalibaculum (asv90; asv18), Colidextribacter (asv124), Oscillibacter (asv163), Parasutterella (asv70) and Lactobacillus (johnsonii_asv2)) in mice fed with Sucrosomial® iron (IS) or ferrous sulfate (FS): directional changes which occur in the microbiome of each mouse after two weeks with the indicated specific diet (B) with respect to the baseline (A).

### SUMMARY OF THE INVENTION

The Applicant found that when a subject is subjected to an iron-based therapy, regardless of the reasons which led the subject to start such therapy (such reasons could be linked to an iron deficiency due to a pathological condition, or, for example, to the need to restore iron levels due to intense sports activity), two cases occur very often depending on the physical condition and health of the subject.

In a first case, one may be dealing with a subject who, besides already per se suffering from a gastrointestinal pathological disorder or condition, also suffers from iron deficiency, which is why the subject in question is on an iron-based therapy. In this situation, the subject taking any iron supplement available on the market, for example based on iron (II), significantly worsens the gastrointestinal pathological condition thereof due to the intake of an iron or an iron salt or an iron ion in an unsuitable form.

In a second case, one may be dealing with a subject who, despite enjoying good health conditions, needs to restore iron levels for reasons that are not of pathological origin or nature, as in the case of a sportsman/sportswoman. Even in this situation, the subject taking any iron supplement available on the market, for example based on iron (II), could cause a gastrointestinal pathological condition due to the intake of an iron or an iron salt or an iron ion in an unsuitable form.

The Applicant found that the formulation thereof, and the composition comprising it, contains an iron or an iron salt or an iron ion in a special and suitable form such not to give rise to changes or alterations in the gut microbiota in favour of a prevalence of harmful pathogenic species. This effect is achieved regardless of whether the formulation of the present invention is administered in the presence of a gastrointestinal infection or a dysbiosis.

An object of the present invention is to provide an iron formulation in solid form for use in a method for the treatment of a gastrointestinal inflammation (i.e. a method for reducing and/or preventing pathological side effects) in a subject subjected to a therapeutic or non-therapeutic treatment of an iron deficiency by administering said iron formulation in solid form of the present invention.

A further object of the present invention is to provide an iron composition for use in a method for the treatment of a gastrointestinal inflammation (i.e. a method for reducing and/or preventing pathological side effects) in a subject subjected to a therapeutic or non-therapeutic treatment of an iron deficiency, wherein said composition comprises said iron formulation in solid form and at least one additive and/or excipient.

A further object of the present invention is to provide a method for the treatment of a gastrointestinal disorder (i.e. a method for reducing and/or preventing pathological side effects) by administering a therapeutically effective amount of said iron formulation or composition, administered in order to treat an iron deficiency, to a subject.

### DETAILED DESCRIPTION OF THE INVENTION

Forming an object of the present invention is an iron formulation in solid form for use in a method for the treatment of a gastrointestinal inflammation (and/or a disease and/or symptom associated therewith) due or not due to an infection, in a subject subjected to a therapeutic treatment (pathological subject) or non-therapeutic treatment (healthy subject) of an iron deficiency by administering said iron formulation of the present invention, wherein said iron formulation reduces or maintains the amount of pathogenic bacteria present in the gut microbiota of the treated subject constant, wherein said pathogenic bacteria preferably belong to the phylum Proteobacteria, and wherein said formulation (in short, formulation of the present invention) comprises or, alternatively, consists of:
(a) an iron in the form of an iron salt or an iron complex or an iron oxide, preferably an iron salt;
(b) at least one phospholipid;
(c) a sucrester or sucrose esters.

Forming another object of the present invention is a solid iron (III) formulation for use in a method for the treatment of a gastrointestinal inflammation in a subject who had been previously subjected to a therapeutic treatment of an iron deficiency by administering to said subject an iron (II) compound or salt, wherein said iron (III) formulation reduces the amount of pathogenic bacteria belonging to the phylum Proteobacteria present in the gut microbiota of the treated subject, and wherein said iron (III) formulation comprises or, alternatively, consists of: (a) an iron (III) salt or complex, (b) at least one phospholipid, (c) a sucrester or sucrose esters. Preferably, said treated subject is a subject with a gastrointestinal inflammation and anaemic, and had been previously treated with a separate and independent treatment based on an iron (II) compound or salt, such as for example iron (II) sulfate, that was unsuccessful given that it did not show any signs of healing or it exacerbated the gastrointestinal inflammation, such as IBD. Preferably, said treatment method is for treating a subject with a gastrointestinal inflammation, for example IBD, and anaemia; where preferably the gastrointestinal inflammation is a relapsed form.

Advantageously, besides reducing or maintaining the amount of pathogenic bacteria present in the gut microbiota of the treated subject constant, said iron formulation in solid form further increases the variety of the gut microbiota of said treated subject.

Said iron formulation in solid form according to the present invention comprising a phospholipid, a sucrester and a plant starch is commercially referred to as Sucrosomial® iron (Sucrosomial® is a trademark registered on behalf of Pharmanutra S.p.A. and Alesco S.r.I.).

Furthermore, forming an object of the present invention is a composition for use in a method for the treatment of a gastrointestinal inflammation and/or of a disease and/or symptom associated therewith (according to any one of the embodiments described in the present invention), in a therapeutic or non-therapeutic treatment of an iron deficiency by administering said composition to a subject, wherein said composition (in short composition of the invention) comprises or, alternatively, consists of said iron formulation of the present invention, according to any one of the described embodiments, and at least one pharmaceutical or food grade additive and/or excipient.

Said method for the treatment of a gastrointestinal inflammation, preferably said inflammation being due to an infection, provides for the administration of a therapeutically effective amount of said iron formulation or composition of the present invention to a subject for treating an iron deficiency and, simultaneously, for preventing the onset or the increase, where already present, of gastrointestinal disorders, in particular, of gastrointestinal infections and/or for reducing said gastrointestinal disorders.

Said gastrointestinal inflammations or diseases or symptoms related therewith may be selected from the group consisting of: intestinal inflammations, gastrointestinal ulcers, chronic gastritis, gastric atrophy, intestinal diverticula, diarrhoea, vomiting, gastrointestinal pain, nausea, constipation.

Examples of said pathogenic bacteria present in the gut microbiota belonging to the phylum Proteobacteria, whose amounts are reduced or maintained constant by administering said iron formulation or composition according to the present invention, are pathogenic bacteria selected from the group comprising or, alternatively, consisting of: Escherichia coli, Helicobacter pylori, Salmonella typhi, Vibrio cholerae, Streptococci.

The iron formulation or composition of the present invention are advantageously for use in the treatment of gastrointestinal inflammations in a subject with, or at risk of contracting, a gastrointestinal infection or disorder, such as for example a subject selected from the group consisting of: a subject with an inadequate nutritional intake, a subject living in inadequate hygiene conditions, a celiac subject, a subject with IBD, a subject subjected to bariatric surgery, a subject with gastritis, a subject with gastric atrophy, a subject with gastroesophageal reflux (or with gastroesophageal reflux disease, GERD).

In the context of the present invention, the expression “inadequate” is used to indicate less than the minimum levels deemed required for the subject to be considered healthy by the person skilled in the art.

Said iron formulations or compositions of the invention may be administered to said subjects for the treatment of an iron deficiency, for example a non-pathological iron deficiency, an anaemia (for example subjects with lower haemoglobin concentration limits with respect to the limits established by the World Health Organisation, WHO), a sideropenia, an iron deficiency in pregnant or breastfeeding women or during the menstrual cycle, an iron deficiency in elderly subjects, or an iron deficiency in subjects with an impaired immune system.

Said iron formulations or compositions of the invention may be administered to said healthy subjects for a non-therapeutic treatment that leads to an increase of iron in the blood (for example sideremia, transferrin, ferritin values) in healthy subjects in order to increase physical performance (for example for sportsmen and sportswomen) or mental performance (for example for learning performance reasons).

Besides (a) an iron salt or complex, (b) at least one phospholipid and (c) a sucrester, the formulation for use of the present invention may preferably further comprise (d) a gelatinised or pre-gelatinised starch of plant origin, preferably rice starch (Oryza sativa) or corn starch, more preferably pre-gelatinised rice starch. Said (a) iron present in the formulation of the present invention (formulation comprising (a), (b), (c) and, optionally, (d)) may be an iron (II) or iron (III) salt or complex, preferably iron (III), selected from an organic or inorganic iron salt, such as chloride, sulfate, pyrophosphate, citrate, bisglycinate, fumarate, gluconate, ascorbate, polymaltosate; preferably said iron consists of iron pyrophosphate (iron (III) pyrophosphate, Fe₄(P₂O₇)₃).

In the context of the present invention, the expression “iron complex” is used to indicate a coordination complex consisting of one or more iron atoms and of one or more atoms, ions or molecules surrounding it, at least partially, without forming an ionic or covalent bond (ligands).

For example, said formulation for use according to the present invention comprises or, alternatively, consists of: (a) iron pyrophosphate, (b) a phospholipid, preferably a lecithin, (c) a sucrester, and optionally (d) pre-gelatinised rice starch.

Said (b) at least one phospholipid present in the formulation of the present invention (formulation comprising (a), (b), (c) and, optionally, (d)) may be a phosphoglyceride selected from the group comprising or, alternatively, consisting of: 1,2-diacyl-phospholipids, 1-alkyl-2-acyl-phospholipids and 1-alkenyl-2-acyl-phospholipids, preferably 1,2-diacyl-phospholipids, such as for example a phosphatidylserine, phosphatidylcholine or lecithin, phosphatidylethanolamine, phosphatidylinositol or phosphatidic acid.

According to a preferred embodiment, in the formulation for use according to the present invention (comprising (a), (b), (c) and optionally (d)) said (b) at least one phospholipid is a lecithin, preferably a lecithin (e.g. E322) selected from the group consisting of: sunflower lecithin, corn lecithin, soy lecithin (for example allergen free).

For example, said formulation for use according to the present invention comprises or, alternatively, consists of: (a) iron pyrophosphate (b) a lecithin, preferably sunflower lecithin, (c) a sucrester, and optionally (d) pre-gelatinised rice starch.

Said (c) sucrester present in the formulation of the present invention (formulation comprising (a), (b), (c) and, optionally, (d)) may be a sucrester E473, preferably a sucrester (E473) comprising from 70% to 90% by weight, with respect to the total weight of the sucrester, at least one sucrose monoester with a fatty acid of plant origin, preferably wherein said fatty acid consists of stearic acid and/or palmitic acid, and optionally (d) a gelatinised or pre-gelatinised starch of plant origin, preferably pre-gelatinised rice starch. According to a preferred example, the formulation for use according to the present invention comprises (a) an iron pyrophosphate, (b) a sunflower lecithin (e.g. E322), (c) a sucrester E473, preferably a sucrester (E473) comprising from 70% to 90% by weight, with respect to the total weight of the sucrester, sucrose monoesters of at least one stearic acid and one palmitic acid, and (d) a pre-gelatinised rice starch.

According to an aspect, the formulation for use according to the present invention (comprising (a), (b), (c) and optionally (d)) comprises said (c) sucrester and said (b) phospholipid, preferably a lecithin, in a [(c):(b)] by weight ratio comprised from 50:1 to 10:1 (for example about 40:1, 35:1, 30:1, 25:1, 20:1, 17:1 or 15:1), preferably from 35:1 to 15:1.

According to an example, the iron formulation in solid form of the present invention (comprising (a), (b), (c) and, optionally, (d)) comprises an iron element (or metal iron or iron (Ill)) in a percentage by weight, with respect to the total weight of the formulation, from 1% to 40% (for example, 5%, 10%, 15%, 20%, 25%, 30% or 35%), preferably from 5% to 20% (for example about 10%-12%, corresponding to about 44%-45% weight/weight of iron pyrophosphate).

According to a further example, the iron formulation in solid form of the present invention (comprising (a), (b), (c) and, optionally, (d)) comprises the components in the following percentages by weight with respect to the total weight of the formulation:
(a) iron element (or metal iron or iron (III)) from 1% to 40% (for example, 5%, 10%, 15%, 20%, 25%, 30% or 35%), preferably from 5% to 20% (for example about 10%-12%, corresponding to about 44%-45% weight/weight of iron pyrophosphate);
(b) phospholipid, preferably a lecithin (for example solid sunflower lecithin (E322)) from 0.05% to 15% (for example, 0.5%, 0.1%, 1%, 2%, 2.5%, 3%, 4%, 6%, 8% or 10%), preferably from 0.1% to 2% (for example about 0.5±0.1% or 1.0±0.1%);
(c) sucrester from 1% to 50% (for example, 3%, 5%, 7%, 10%, 12%, 15%, 17%, 20%, 25%, 30%, or 40%), preferably from 10% to 25%, for example about 17%-18%±1%); and optionally,
(d) starch of plant origin, preferably pre-gelatinised rice starch, from 10% to 85% for example, 15%, 20%, 30%, 35%, 40%, 50% or 60%), preferably from 25% to 50% (for example about 37-38±1%).

The term lecithin is used to indicate a class of chemical compounds present in animal and plant tissues (particularly in the egg yolk). Lecithin is a natural emulsifier and it has antioxidant properties.

From a chemical point of view, a lecithin consists of a phosphatidylcholine ((R)-1-Oleoyl-2-palmitoyl-phosphatidylcholine) or it comprises—as primary component—at least one phospholipid, preferably phosphatidylcholine. A phosphatidylcholine is a phosphoglyceride in which phosphatidic acid is esterified with choline. Phosphatidic acids represent the simplest phosphoglycerides formally produced by the esterification of glycerol in position 1 and 2 with fatty acids and in position 3 with orthophosphoric acid.

Said (b) phosphatidylcholine or lecithin comprised in the formulation of the present invention (together with (a), (c) and optionally (d)) is preferably selected from sunflower lecithin, corn lecithin and soy lecithin, more preferably a lecithin in solid form (e.g. powder or granules) such as a solid lecithin E322, even more preferably a solid sunflower lecithin (e.g. E322), for example an allergen free solid sunflower lecithin E322 (preferably in form of powder or granules). The term “allergen free” means that it does not have any allergen residues. The term “E322” indicates that lecithin is a food additive (emulsifier) permitted by European legislation and regulated by the Italian Ministerial Decree No. D.M. 1996.

Directive 2008/84/EC of 27 Aug. 2008 (published in the Official Journal of the European Union No L253) lays down the purity criteria which a lecithin must meet in order to be considered to meet food quality standards (lecithin E322): insoluble in acetone (i.e. active component) 60% minimum; moisture: 2% maximum; acidity number: 35 maximum; peroxides number: 10 maximum; insoluble in toluene (i.e. impurities): 0.3% max.

A (b) phosphatidylcholine or lecithin in form of powder or granules (preferably sunflower lecithin E322) comprised in the formulation of the invention may have, for example, a water content in a percentage by weight comprised from 1.5% to 4.5% (for example 2%, 2.5%, 3.5% or 4%) with respect to the weight of the lecithin; and/or a glucose content in a percentage by weight comprised from 20% to 60% (for example 30%, 40%, 45% or 50%) with respect to the weight of the lecithin. An example of (b) sunflower lecithin E322 (powder or granules) which can be used in the context of the present invention may have the following composition (chemical/physical analysis, % weight/weight): sunflower lecithin from 40% to 50%, carbohydrates from 40% to 50% (for example about 42%), proteins from 6% to 10%, ashes from 3% to 8%, moisture from 2% to 5% and another flowing agent from 0.5% to 1.5%.

According to an aspect, said (b) lecithin comprised in the formulation of the present invention (together with (a), (c) and, optionally, (d)) does not comprise, or alternatively consist of a decomposed or hydrolysed lecithin, and it does not comprise, or alternatively consist of an enzymatically decomposed or hydrolysed lecithin (that is, decomposed or hydrolysed enzymatically).

The (c) sucrester (or sucrose esters or carbohydrate fatty acid esters, as synonyms of sucrester) comprised in an embodiment of the formulation of the present invention (together with (a), (b) and, optionally, (d)) may be a sucrester E473, preferably a sucrester (E473) comprising in percentage by weight, with respect to the total weight of the sucrester, from 50% to 95% (for example 60%, 70%, 80%, 85% or 90%, preferably from 70% to 90%) of monoesters obtained by esterifying sucrose with one or more fatty acids of plant origin, preferably stearic acid and/or palmitic acid. The abbreviation “E473” indicates that sucrester is a food additive (emulsifier) permitted by European legislation and regulated by the Italian Ministerial Decree No. D.M. 1996.

Sucresters are generally obtained by esterifying fatty acids or by trans-esterifying the methyl esters of fatty acids with carbohydrates (also called saccharides); the carbohydrates used are sucrose (monosaccharide) and the polysaccharides (hence the name “sucrose esters of fatty acids”). The chemical/physical properties of these compounds depend on the number and the type of esterified fatty acids. Sucresters are essentially emulsifiers and they are added to the compositions to increase the stability of a hydrophilic phase with a lipophilic phase.

An example of (c) sucrester (E473) that can be used in the context of the present invention is a sucrester having a hydrophilic-lipophilic balance (HLB) value comprised in the range from 14 to 18 (for example 15, 16 or 17) and/or having the following composition (% weight/weight): total ester content of at least 90%, of which at least 70% of monoesters obtained by esterifying sucrose with one or more fatty acids of plant origin, preferably stearic acid and/or palmitic acid; free fatty acid content (e.g. oleic acid) not higher than 3%; free sucrose content not higher than 2%; moisture not higher than 4%; acidity value not higher than 5. An example of (c) commercial sucrester is sucrose esters SP70 produced by Chimab S.p.A—Italia.

According to an aspect, said (c) sucrester comprised in the formulation of the present invention (together with (a), (b) and, optionally, (d)), does not comprise, or alternatively consist of a polyglycerol fatty acid ester.

Said (d) pre-gelatinised rice starch, optionally comprised in the formulation of the present invention (together with (a), (b) and (c) and optionally (e)) may have, for example, the following chemical/physical characteristics: moisture not higher than 7%; protein content not higher than 1%; ash content not higher than 1%; pH (solution 10%) comprised from 5.5 to 7.5, density 0.40-0.48 g/cm 3; minimum starch content at 97% and fat content not higher than 0.1%. An example of commercial pre-gelatinised rice starch that can be used in the context of the present invention is AX-FG-P produced by Reire Srl—Italia.

The components (a), (b), (c) and (d) described in the present invention may be prepared according to techniques and equipment known to the person skilled in the art and they are available on the market.

Said composition of the present invention may be a pharmaceutical composition, a composition for medical devices (Medical Device Regulation (EU) 2017/745 (MDR)), a dietary supplement and/or a food for special medical purposes (FSMP).

Said at least one pharmaceutical or food grade additive and/or excipient, comprised in the composition of the present invention together with the iron formulation in solid form of the invention, consists of a substance devoid of therapeutic activity suitable for pharmaceutical or food use selected from ancillary substances known to the person skilled in the art such as for example diluents, solvents, solubilizers, thickeners, sweeteners, flavour enhancement agents, dyes, lubricants, surfactants, antimicrobials, antioxidants, preservatives, anti-caking agents, fillers, charging agents, pH stabilising buffers and mixtures thereof. Non-limiting examples of such substances are hydroxymethyl cellulose, dye E171, vegetarian magnesium stearate, magnesium salt of fatty acids, silicon dioxide, microcrystalline cellulose.

Besides the iron formulation of the present invention and additives/excipients, the composition of the present invention may further comprise (e) at least one vitamin, preferably selected from the group comprising or, alternatively, consisting of: (e-i) a vitamin C, (e-ii) at least one vitamin of group B, preferably vitamin B6 and/or B9 and/or B12, (e-iii) a vitamin of group D, preferably vitamin D3, (e-iv) a vitamin E, and mixtures thereof; preferably (e-i) vitamin C and/or (e-ii) at least one vitamin of group B.

Said (e) at least one vitamin may be present in the composition in an amount from 500% RDA to 300% RDA, for example 100% RDA, 150% RDA, 200% RDA or 250% RDA (RDA: Recommended dietary Allowance).

Said (e) at least one vitamin comprised in the composition of the present invention may be a Sucrosomial® vitamin, that is a formulation in solid form comprising said (e) at least one vitamin, (b) a phospholipid (for example a lecithin), (c) a sucrester, and optionally (d) a plant starch (for example pre-gelatinised rice starch), similarly to any of the embodiments described for the Sucrosomial® iron formulation in solid form of the present invention.

Examples of compositions according to the present invention comprising a Sucrosomial® iron and at least one Sucrosomial® vitamin are:
iron and at least one vitamin selected from: vitamin C, vitamin D3, a vitamin B (preferably B12), vitamin E, and a mixture thereof; iron and vitamin C; iron and vitamin D3; iron and vitamin C and vitamin D3; iron and vitamin C e vitamin B12; iron and vitamin D3 and vitamin B12; iron and vitamin C and vitamin D3 and vitamin B12.

The compositions of the present invention, according to any one of the described embodiments, are preferably formulated in solid form, for example in solid form as is or in mouth dissolvable solid form (or orosoluble, L a that dissolve in the oral cavity) or water dispersible solid form (i.e. which dissolve in a water-based liquid), such as for example powder, granules, microgranules, flakes, tablets or capsules.

Alternatively, the compositions of the present invention, according to any one of the described embodiments, may be in liquid form, for example a solution, a dispersion or a suspension of a solid in a liquid, or in semi-solid form, for example a cream, a gel or a soft-gel.

The formulations or compositions of the present invention are preferably formulated for oral administration, for example in solid form as is or mouth dissolvable or water dispersible.

The compositions of the invention in form of tablet may have a weight comprised in the range from 200 mg to 2000 mg, for example a hard tablet from 800 mg to 1000 mg. Said tablets may be coated or filmed with one or more coating layers or films capable of going past the gastric barrier. Said coating may comprise bee wax or a sugar-based solution.

The compositions of the invention in form of capsule may have a weight comprised in the range from 100 mg to 1500 mg, more preferably of about 800 mg; said capsule may be of a rigid gelatin or soft gelatin or soft-gel.

The iron formulation of the present invention (Sucrosomial® iron, according to any one of the described embodiments) may be prepared according to a first method as described in the patent document WO 2014/009806 A1 from page 7 line 1 to page 8 line 20 (incorporated in the present application for reference) or according to a second method described in the patent document WO 2014/009806 A1 from page 8 line 22 to page 10 line 21 (incorporated in the present application for reference).

For example, a method for the preparation of an iron formulation of the present invention comprises the steps of: (1) preparing an iron salt or complex in form of powder or granules; (2) mixing said iron salt or complex with a phospholipid, preferably lecithin (for example, sunflower lecithin) and with at least one sucrester, to obtain a mixture of step 2 or the formulation of the invention; advantageously, said lecithin is not a hydrolysed or enzymatically hydrolysed lecithin; (3) optionally, mixing said mixture of step 2 with a starch of plant origin (for example, pre-gelatinised rice starch), to obtain the formulation of the invention; (4) optionally after step 2 or after step 3, sieving, for example with a sieve having a nominal sieve opening comprised from 150 μm to 1400 μm (for example 180, 212, 250, 300, 355, 425, 500, 600, 710, 850, 1000, or 1180 microns), preferably from 600 μm to 850 μm, for example 710 μm (25 US Mesh).

Preferably, said mixing steps 2 and/or 3 are carried out at room temperature (from 15° C. to 30° C., preferably at about 25° C.) for a period of time comprised from 10 minutes to 120 minutes, preferably from 15 minutes to 60 minutes, for example about 30 minutes.

According to a preferred aspect, said mixing steps 2 and/or 3 are carried out in the absence of solvent (dry mixing) and/or said process for preparing an iron formulation of the present invention does not comprise a spray-dry step.

Furthermore, said step 2 for mixing the iron with a phospholipid and at least one sucrester may be divided into two sub-steps, wherein the first sub-step provides for mixing the iron with a phospholipid and the second sub-step provides for mixing the mixture obtained from the first sub-step with said at least one sucrester.

Said method for preparing an iron formulation of the present invention is carried out using the equipment known to the person skilled in the art and, for example, by mixing the components (a), (b), (c) and, optionally, (d) according to the percentages by weight defined in the context of the present invention.

Said at least one Sucrosomial® vitamin optionally comprised in the composition of the present invention together with the iron formulation of the present invention, may be prepared according to the method for the preparation of said iron formulation of the present invention as described in the present description, using said at least one vitamin instead of iron.

In the context of the present invention, the expression “subject/s” is used to indicate mammals (animals and humans), preferably human subjects, men and women, for example including subjects in paediatric age, adults, elderly subjects, subjects with diseases and subjects usually active in sports activities.

The expression “therapeutically effective amount” is used to indicate the amount of iron formulation which elicits the biological or medicinal response in a tissue, system or subject which is sought and defined by a person skilled in the art.

Unless otherwise specified, the expression formulation or composition comprising a component in an amount “comprised in a range from x to y” is used to indicate that said component may be present in the formulation or composition in all the amounts existing between the extremes of said range, even though not specified, extremes of the range comprised.

Unless otherwise specified, the content of a component in a formulation or composition refers to the percentage by weight of that component with respect to the total weight of the formulation or the composition. Unless otherwise specified, the indication that a formulation or composition “comprises” one or more components means that other components—besides the one, or the ones, indicated specifically—can be present and the indication that a formulation or composition “consists” of determined components means that the presence of other components is ruled out.

Embodiments of the present invention (FRn) are reported below.
FR1. An iron formulation for use in a method for the treatment of a gastrointestinal inflammation or a disease or a symptom related therewith, in a subject subjected to a therapeutic or non-therapeutic treatment of an iron deficiency by administering said iron formulation to said subject,
wherein said iron formulation reduces the amount of pathogenic bacteria belonging to the phylum Proteobacteria present in the gut microbiota of the treated subject, and
wherein said iron formulation comprises or, alternatively, consists of:
(a) an iron salt or complex,
(b) at least one phospholipid,
(c) a sucrester or sucrose esters.
FR2. The formulation for use according to FR1, wherein said treatment method is for reducing said gastrointestinal inflammation; preferably said inflammation is due to an infection.
FR3. The formulation for use according to FR1 or FR2, wherein said pathogenic bacteria belonging to the phylum Proteobacteria are selected from the group comprising or, alternatively, consisting of: Escherichia coli, Helicobacter pylori, Salmonella typhi, Vibrio cholerae Streptococchi.
FR4. The formulation for use according to any one of FR1-FR3, wherein said iron formulation increases or modifies the variety of the gut microbiota of the treated subject.
FR5. The formulation for use according to any one of FR1-FR4, wherein said formulation further comprises (d) a gelatinised or pre-gelatinised starch of plant origin; preferably rice starch or corn starch; more preferably pre-gelatinised rice starch.
FR6. The formulation for use according to any one of FR1-FR5, wherein said (b) at least one phospholipid consists of a lecithin; preferably a lecithin (E322) selected from the group consisting of: sunflower lecithin, corn lecithin, soy lecithin and mixtures thereof.
FR7. The formulation for use according to any one of FR1-FR6, wherein said (c) a sucrester or sucrose esters consists of sucrester E473; preferably a sucrester (e.g. E473) comprising from 30% to 95% by weight, preferably from 50% to 85% by weight, with respect to the total weight of the sucrester, at least one sucrose monoester with a fatty acid of plant origin, preferably wherein said fatty acid consists of stearic acid and/or palmitic acid.
FR8. The formulation for use according to any one of FR1-FR7, wherein said formulation comprises or, alternatively, consists of:
(a) iron pyrophosphate,
(b) sunflower lecithin;
(c) sucrester or sucrose esters; and
(d) pre-gelatinised rice starch;
preferably, wherein a [(c):(b)] by weight ratio between said (c) sucrester or sucrose esters and said (b) phospholipid or lecithin is comprised from 50:1 to 10:1, preferably from 40:1 to 10:1, more preferably from 30:1 to 15:1.
FR9. The formulation for use according to any one of FR1-FR8, wherein said gastrointestinal infection and/or disease or symptom related therewith are selected from the group consisting of:
intestinal inflammations, gastrointestinal ulcers, chronic gastritis, gastric atrophy, intestinal diverticula, diarrhoea, vomiting, gastrointestinal pain, nausea, constipation.
FR10. The formulation for use according to any one of FR1-FR9, wherein said subject is a subject having or at risk of a gastrointestinal infection;
preferably wherein said subject having or at risk of a gastrointestinal infection is selected from the group consisting of: subject with inadequate nutritional intake, subject living in inadequate hygiene conditions, celiac subject, subject with IBD, subject subjected to bariatric surgery, subject with gastritis, subject with atrophic gastritis, subject with gastroesophageal reflux disease (GERD).
FR11. A composition for use according to any one of FR1-FR10, wherein said composition comprises or, alternatively, consists of:
the formulation according to any one of the preceding claims, and at least one pharmaceutical or food grade additive and/or excipient.

### Examples

Reported below are some examples of iron formulation in solid form according to the present invention.

TABLE A

| Example 1 | Amt (g/) per 100 g |
|---|---|
| Ultra-fine iron (III) pyrophosphate | 40-50 |
| Pre-gelatinised rice starch (Oryza sativa L.) | 35-42 |
| Saccharide esters of fatty acids (sucrester) | 15-19 |
| Plant lecithin for example sunflower | 0.5-1.5 |
| Total | 100.00 |

TABLE A.1

| Example 1.1 | Amt (g/) per 100 g |
|---|---|
| Ultra-fine iron (III) pyrophosphate | 44.760 |
| Pre-gelatinised rice starch (Oryza sativa L.) | 37.520 |
| Saccharide esters of fatty acids (sucrester) | 17.140 |
| Plant lecithin for example sunflower | 0.580 |
| Total | 100.00 |

TABLE A.2

| Example 1.2 | Amt (g/) per 100 g |
|---|---|
| Ultra-fine iron (III) pyrophosphate | 40.760 |
| Pre-gelatinised rice starch (Oryza sativa L.) | 38.520 |
| Saccharide esters of fatty acids (sucrester) | 19.240 |
| Plant lecithin for example sunflower | 1.480 |
| Total | 100.00 |

TABLE A.3

| Example 1.3 | Amt (g/) per 100 g |
|---|---|
| Ultra-fine iron (III) pyrophosphate | 41.760 |
| Pre-gelatinised rice starch (Oryza sativa L.) | 40.520 |
| Saccharide esters of fatty acids (sucrester) | 16.640 |
| Plant lecithin for example sunflower and/or soy | 1.080 |
| Total | 100.00 |

### EXPERIMENTAL PART

### Part I

### 1. Purpose of the Study

Determining the effect of Sucrosomial® iron also referred to as Sideral® RM (iron (III) according to the present invention, according to Table A.1) on the gut microbiome of mice and comparing it with the effect of ferrous sulfate FeSO4 (comparative iron (M. The comparison was made using the same iron element in the salt used (iron in ionic form as Fe(III) in one case and Fe(II) in the other).

### 2. Methodology

Thirty C57BL/6 male mice at four weeks of age were subjected to an iron deficient diet (based on the AIN-93G rodent diet, ˜1 mg kg of iron). All mice were bred in the same environment (QIMR Berghofer Animal Facility) to minimise the inherent variability in the microbiome between individual mice.

At six weeks of age (i.e. two weeks with an iron deficient diet), faeces were collected for microbiome analysis. This was the control sample for each mouse and it represents the basic microbiome of that particular animal when the lumen is iron deficient. At this point (six weeks of age, two weeks with a deficient diet), mice are assumed to have reduced iron reserves but they are not anaemic, thus avoiding any potential additional effects of anaemia on the microbiome, as this could complicate the analysis.

After faecal collection at six weeks of age, mice were switched from an iron deficient diet to a diet containing 50 mg/kg of iron such as ferrous sulfate (iron (II) sulfate) or Sucrosomial® iron (ten mice per group).

The remaining ten mice were a control group that remained on the deficient diet.

Faecal samples were then collected from each mouse at eight weeks of age (i.e. two weeks with diets containing iron), the mice sacrificed and the tissues collected for analysis. Liver and serum samples were collected from each animal after euthanasia and stored at −80° C. Haematological parameters were evaluated immediately after euthanasia using a “Coulter Ac-T diff Hematology Analyzer” (Beckman Coulter, Gladesville, NSW, Australia). Serum iron parameters, analysed using the Iron/TIBC Reagent Kit (Pointe Scientific, Canton, MI), and iron levels in tissues were determined as described above [Frazer, D. M., et al. Cell Mol Gastroenterol Hepatol 3, 410-421 (2017)]. For the analysis of iron and haematological parameters, the statistical differences between the groups were calculated using the unidirectional ANOVA followed by the Tukey post-hoc test for the groups with equal variance or by the Games-Howell post-hoc test for those with unequal variance. Significant differences in variance were identified using the Levene test. DNA was extracted from the faecal samples and the microbial profile (QIMR Berghofer Medical Research Institute) was determined.

### Groups of Mice Under Study:

ID: control group, maintained on iron deficient diet (about 1 mg/kg iron).
FS: group fed with diet containing 50 mg/kg of iron in the form of ferrous sulfate (FS abbreviation for ferrous sulfate)—Fe(II).
SI: group fed with diet containing 50 mg/kg of iron in the form of Sucrosomial® iron (SI abbreviation for sucrosomial iron)—Fe(III).

### 3. Results

3.1 the Iron Status of Mice Fed with Diets Based on Sucrosomial® Iron or Ferrous Sulfate.

Before determining the effect on the microbiome, the iron status was analysed in the three groups of rats under analysis as identified in section 2 (ID, FS and SI).

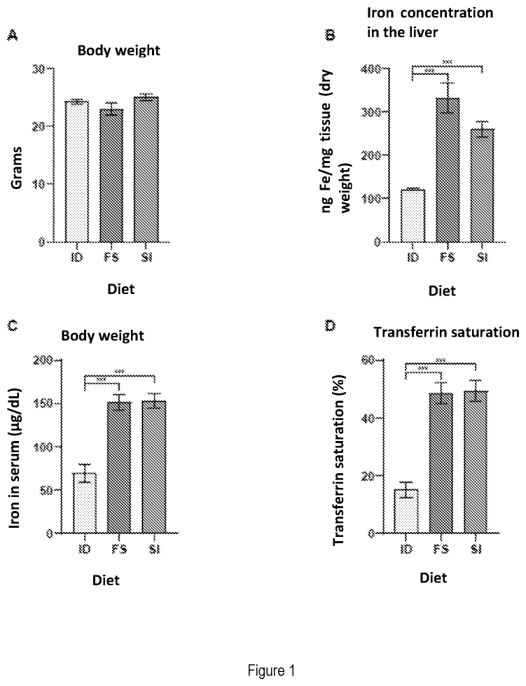

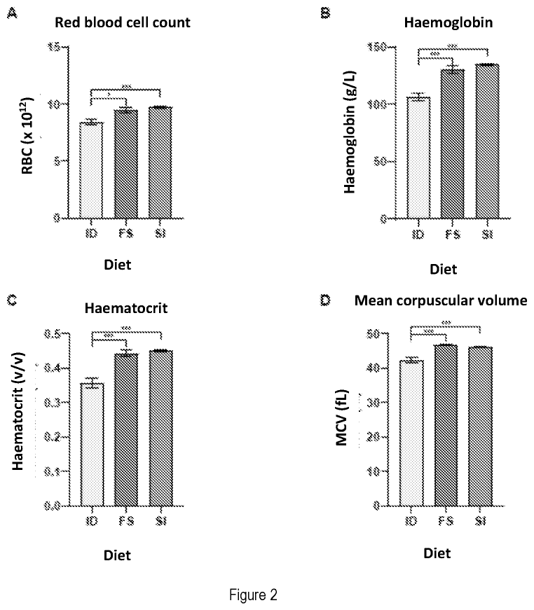

After being sacrificed at the age of eight weeks, iron levels in the liver and in the serum, as well as the haematological parameters of mice of the three groups under analysis (ID, FS and SI) were examined. All data of FIGS. 1A-D represent mean±SEM with 10 mice per group (* P<0.05, *** P<0.005).

There were no significant differences in body weight in the three groups (ID, FS and SI; FIG. 1A). There were no significant differences in iron concentration in the liver, in serum iron levels, in transferrin saturation, in red blood cell count, in haemoglobin, haematocrit or mean corpuscular volume between the groups treated with Sucrosomial® iron and ferrous sulfate (FIGS. 1B-D, 2A-D). However, in any case, the average for the iron deficient group (ID, control group) was significantly lower than the groups with diets containing iron (FS and SI) (FIGS. 1B-D, 2A-D). These animals (ID, control group) had followed an iron deficient diet for four weeks, so it is not unexpected that they would show signs of iron deficiency.

Having determined that the iron status was similar in the groups treated with iron (Sucrosomial® iron (SI) or ferrous sulfate (FS)), modifications to the microbiome in these animals were examined.

### 3.2. Overall Microbial Composition—Beta Diversity

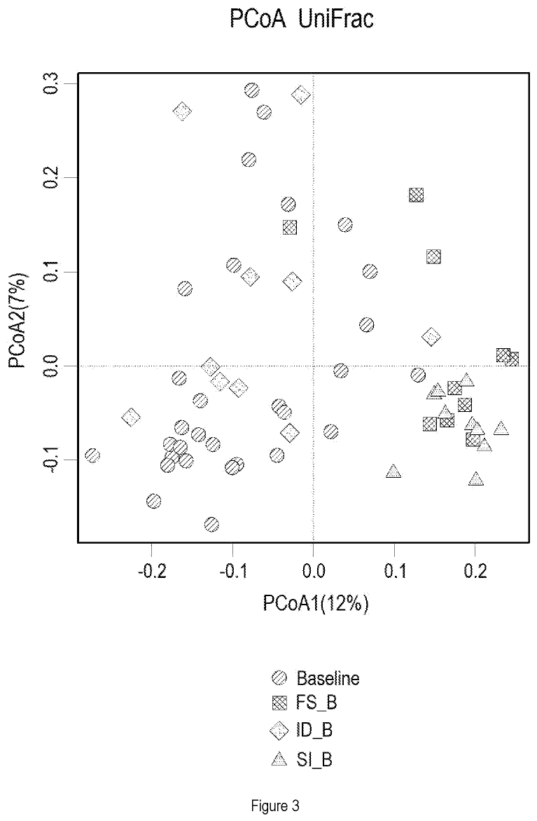

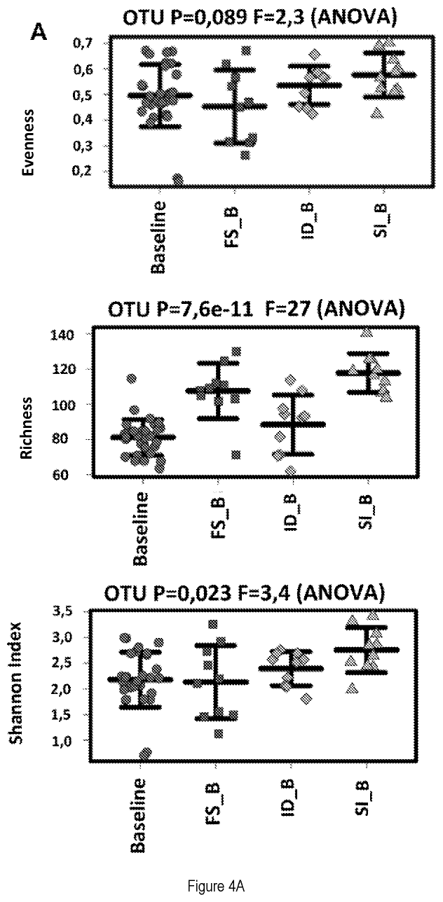

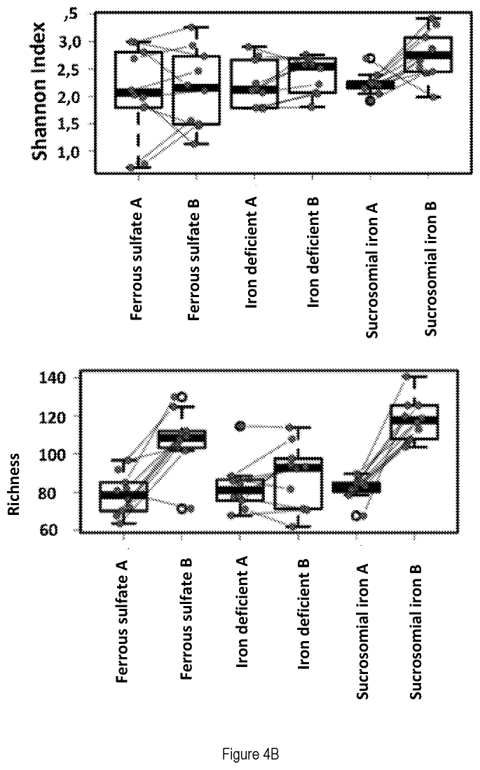

Beta diversity compares the overall microbial composition between the samples, i.e. it shows how the different samples are based on their identified microbial profile. Principal Coordinates Analysis (PCoA) sorts samples based on their overall microbial community in a two-dimensional space. Each symbol in FIG. 3 represents a sample with the form indicating the diet and the time point (i.e. the baseline or after another two weeks on the specific diets). The reference samples are those taken from each mouse after the first two weeks of iron deficient diet (baseline). The distance between each sample shows how similar/dissimilar the samples are with respect to the other samples (that is the more separated the more dissimilar they are). The data show that samples taken from mice fed with one of the iron-rich diets (ferrous sulfate (FS) or sucrosomial iron (IS)) are largely grouped together and were able to be distinguished from samples taken from mice on iron-deficient diets (ID). This indicates that diets containing iron have been able to alter the overall microbial diversity. There was observed both a cage effect (that is those animals housed together had more similar microbiomes) and a mouse effect (that is it was observed that the microbiome changes over time in the same mouse even with an iron deficient diet). These effects are not unusual when it comes to mice and they were corrected in any subsequent analysis.

### 3.3. Overall Microbial Composition—Alpha Diversity

Alpha diversity measures microbial diversity within a single sample. Several parameters are used to measure sample diversity. The richness refers to the number of different microbial sequences detected and it is an estimate of the number of bacterial strains in the sample. Evenness tells us how evenly bacteria are distributed in a sample, that is if there is a dominant species. Shannon index is a combination of the richness and evenness of each sample. It is generally accepted that an increase in Shannon's index is better for health. In our samples, diversity changed significantly using unpaired statistics (ANOVA). Reference samples (baseline) showed the lowest richness, followed by the iron deficient group (ID) and iron-rich diets (FS and SI). The Shannon index was lower in ferrous sulfate-fed mice and higher in sucrosomial iron-fed mice (FIG. 4A). This indicates that the overall intestinal microbial health was improved with the diet containing Sucrosomial® iron (SI) with respect to the iron deficient diet (ID) and the diet containing ferrous sulfate (FS). In mixed effect regression analysis with diversity indices (Shannon, richness, chao 1 (another richness), evenness index), as result with “mice” and “cage” as random effects, “diet” as the independent variable and “time” as fixed effect, change due to the individual heterogeneity of the gut microbiota was then carried out (FIG. 4B). We have seen a significant increase in Shannon index by switching to the diet containing sucrosomial iron alone, once again suggesting that this diet produces a healthier gut microbiome.

### 3.4. Changes at Phylum Level

Although diversity indices measure the overall health of the microbiome, it is useful to examine the types of bacteria present. Although many sequences obtained by 16S sequencing have not yet been assigned to a particular bacterial species, many of the amplicons can be grouped into taxonomic classifications that provide information on the changes in progress. Taxonomic classifications are based on a taxonomic classification hierarchy, with very general top ranks (e.g. phylum) and very specific bottom ranks (e.g. genus). For all of the following analyses, P values were obtained from a mixed effect regression model with abundance of taxon as a result, “mice” and “cage” as random effects, “diet” as independent variable and “time” as fixed effect. P values were adjusted using Bonferroni correction to avoid false positives. A P value lower than 0.05 was deemed significant.

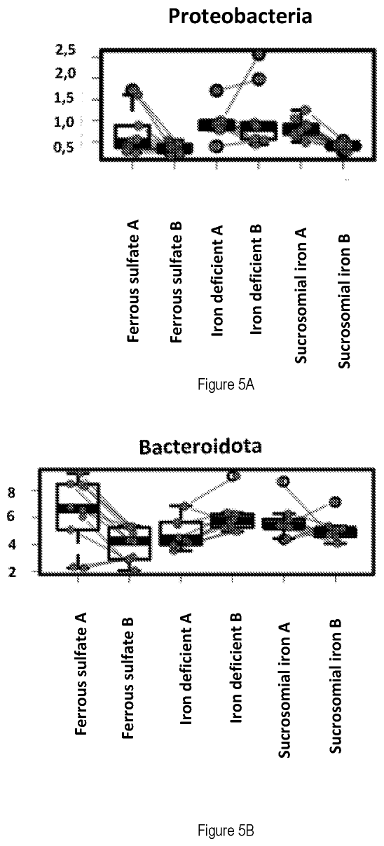

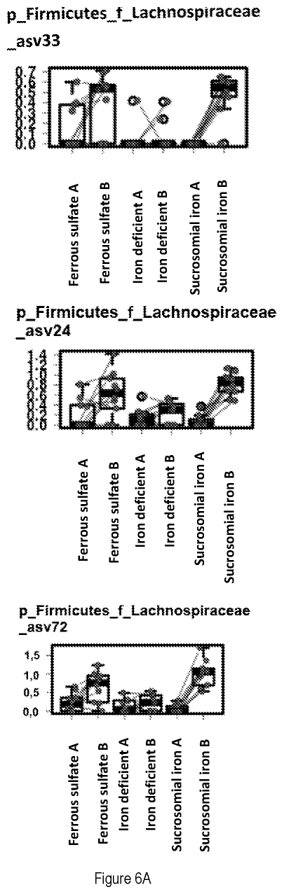

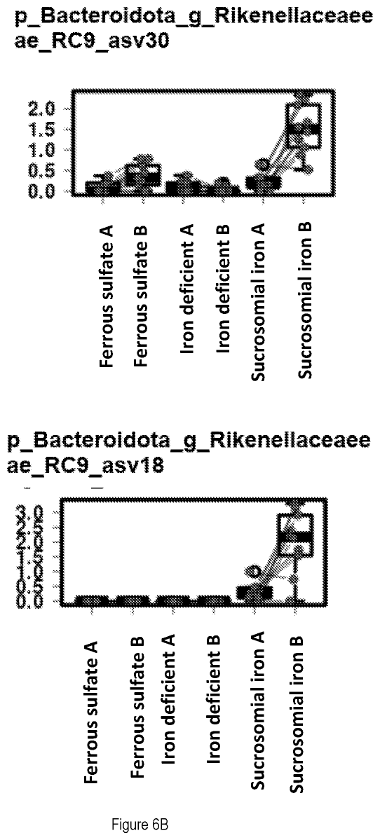

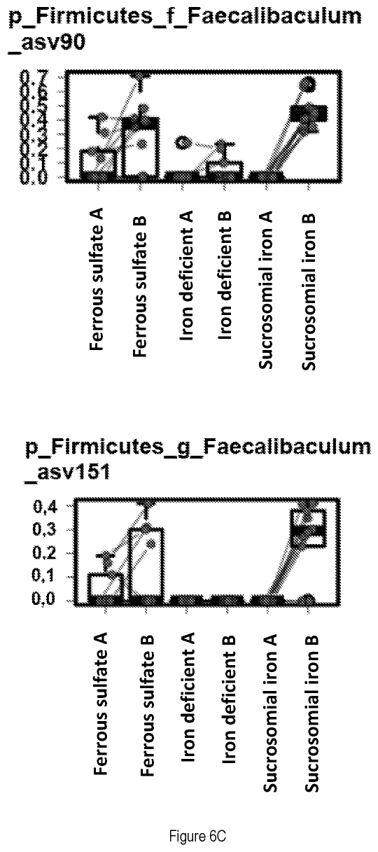

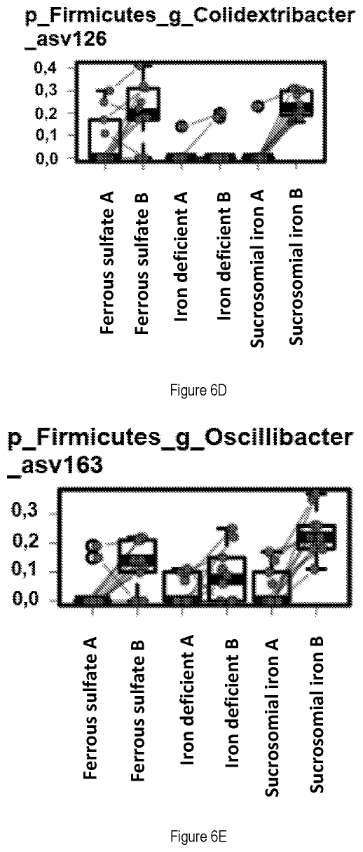

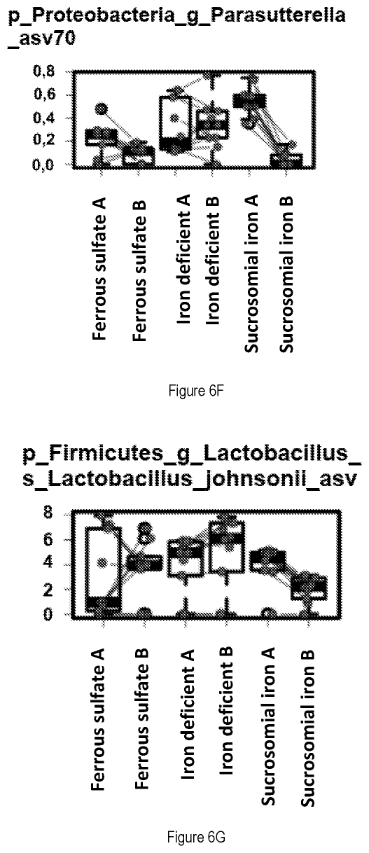

At the phylum level, Proteobacteria were significantly decreased in the group with diet containing Sucrosomial® iron (SI), but not in the iron deficient diet group (ID) or the group with diet containing ferrous sulfate (FS) (FIG. 5). Proteobacteria are a phylum containing many pathogens such as Escherichia, Enterobacter and Salmonella. Previous studies have shown that iron supplements may increase the prevalence of gastrointestinal pathogens in areas where said pathogens are endemic. Although these pathogens were not present in our animals, the decrease in Proteobacteria in the Sucrosomial® iron (SI) group entails a protective intestinal effect of this iron supplement and suggests that Sucrosomial® iron is a better option with respect to other iron supplements in areas where intestinal pathogens are endemic.

The members of the phylum Bacteroidota were significantly reduced in mice fed with the diet containing ferrous sulfate (FS), but they remained unchanged in the diet containing Sucrosomial® iron (SI) (FIG. 5). Significant increases in the iron deficient diet were observed. Although the reasons for these changes are unclear, Bacteroidota is one of the main phyla that inhabits the bowel and it is involved in the production of short chain fatty acids. Reductions in Bacteroidota have been linked to obesity.

### 3.5. Changes at the Amplicon Single Variant (ASV) Level

ASVs represent the actual sequenced amplicon. Due to the complexity of the microbiome, many were not fully characterised at species level. Various ASVs increased in the group fed with the diet containing Sucrosomial® iron (SI), including Lachnospiraceae spp, Rikenellaceae RC9, Faecalibaculum, Colidextribacter and Oscillibacter (FIGS. 6A-6E). These bacterial groups are generally associated with a healthy microbiome due to their ability to produce short chain fatty acids.

Faecalibaculum has been shown to have a protective effect against intestinal tumours. This is interesting because studies on rodents have shown that ferrous sulphate (FS) can promote the development of intestinal cancer.

In contrast, the bacteria of the genus Parasutterella decreased in the group of Sucrosomial® iron (IS) (FIG. 6F). Although the species Parasutterella are also associated with irritable bowel syndrome and chronic intestinal inflammation, bacterial strains belonging to this genus are considered normal members of the gut microbiome.

Lactobacillus decreased at the genus level only in animals treated with Sucrosomial® iron (SI) (FIG. 6G). Although the species Lactobacillus are considered beneficial, this decrease should not be considered a negative effect of sucrosomial iron, given that this genus represents one of the few groups of organisms that do not require iron for survival. Therefore, the results probably represent a proportional increase of the species Lactobacillus during the first two weeks of iron deficient diet, when the growth of other bacteria requiring iron would be suppressed. Switching to a diet containing Sucrosomial® iron (SI) would allow the proliferation of other bacterial species, increasing the overall diversity and health of the gut microbiome. The resulting increase in competition would reduce Lactobacillus to a more normal level.

The observation that there was no similar decrease in Lactobacillus in animals following a diet based on ferrous sulfate (FS) likely reflects the lack of diversity in this group, with less competition allowing Lactobacillus levels to remain high.

The lack of diversity indicates a less healthy microbiome.

### 4. Conclusions

Overall, changes in gut microbiome induced by a diet containing Sucrosomial® iron (IS) are beneficial, with an increase in microbial diversity with respect to a diet containing ferrous sulfate (FS) or an iron deficient diet. An increase in microbial diversity is generally believed to produce a healthier microbiome. More commonly, an increase in diversity is associated with a decrease in pathogenic bacteria belonging to the phylum Proteobacteria.

Accordingly, a decrease of said phylum Proteobacteria was observed in the Sucrosomial® iron (SI) diet. The decrease of Proteobacteria, a phylum containing many pathogenic bacterial strains, with the diet based on Sucrosomial® iron is potentially beneficial, given that iron-based supplements have been shown to increase the risk of intestinal infections, reducing the effectiveness thereof in areas where intestinal pathogens are endemic.

Sucrosomial® iron (IS) also favoured the growth of beneficial bacteria known for the production of short chain fatty acids.

Based on the above, the results suggest that Sucrosomial® iron has more beneficial effects on gut microbiome with respect to ferrous sulfate (FS) in a murine model.

### Part II—Study Design

### Introduction

Iron is an essential component present in haemoglobin in red blood cells and myoglobin in muscles, and it is also essential for the function of many cellular activities. However, iron may also be toxic given that it catalyses the production of reactive oxygen species, therefore iron homeostasis is well regulated in mammals, especially in duodenum where iron is mainly absorbed. The gut microbiota represents the totality of the microorganisms present in the gastrointestinal tract (GIT). The gut microbiota includes all the bacteria—commensal and pathogenic—resident in the gastrointestinal tract GIT. The gut microbiota plays an important role in the absorption of minerals and nutrients, in the synthesis of enzymes, vitamins and amino acids and in the production of short-chain fatty acids (SCFAs). However, there is a potential risk that these mechanisms may be impaired as a result of an alteration in the microbial composition, also known as dysbiosis.

The gut microbiota encounters different concentrations of unabsorbed iron that come from the diet, supplements or iron-based drugs administered through the oral route. Iron is essential for bacteria given that it functions as a cofactor in iron-containing proteins involved in redox reactions, metabolic models and in the mechanisms present in the electron transport chain, so that the iron status in the host and the availability of iron present in the diet can modify this microbial medium.

Administration of iron through the oral route may increase the growth and virulence of intestinal pathogenic bacteria, causing diarrhoea and intestinal inflammation. On the other hand, iron deficiency also changes the microbial composition.

One way to combat iron deficiency is by supplementing with iron-based products; however, this mode is not always effective and it can give rise to serious side effects.

In order to overcome the onset of side effects due to supplementation with iron-based products through the oral route there is a need for novel formulations containing iron for oral use that are effective at protecting iron as well as increasing intestinal absorption, so as to reduce dosage and side effects.

The composition of the present invention comprising or, alternatively, consisting of: (a) an iron salt or complex (iron (III) pyrophosphate), (b) at least one phospholipid, and (c) a sucrester or sucrose esters has the advantage of having an iron (III) pyrophosphate protected by a sucrester and phospholipid matrix. The composition of the present invention is mostly in the form of a vesicle-like structure overcoming conventional iron absorption models (in-vitro studies). Due to this behaviour in the gastrointestinal tract, the composition of the present invention is well tolerated and highly bioavailable as compared to conventional iron salts such as iron (II) sulfate.

Given that oral administration of iron may modify the composition of the gut microbiota, due to the ability of the microbes to metabolise iron by increasing the proliferation of pathogenic bacteria, the purpose of this experimental part is to investigate modifications of the faecal microbiome in iron deficient subjects to whom iron (III) pyrophosphate, according to the present invention, and iron (II) sulfate, were administered.

### Primary Purpose of the Study

Evaluate the modifications of the faecal microbiota during supplementation with two different iron formulations (Sucrosomial® Iron (composition of the present invention containing iron (III) pyrophosphate) versus Iron (II) sulfate).

### Secondary Purpose of the Study

Evaluate faecal calprotectin concentration.

### First Endpoint of the Study

The first target of this study is to compare the composition of the faecal microbiota in terms of different bacterial populations, the abundance thereof and biodiversity, to evaluate whether different iron formulations can lead to different microbiota compositions. Baseline microbiota, collected prior to treatment, will be compared against three different time points during and after treatment.

### Second Endpoint of the Study

The second target of this study is focused on the evaluation of a possible faecal calprotectin change and the better understanding of a correlation with faecal microbiota changes.

### Population Under Study

20 subjects, both anaemic and non-anaemic healthy subjects but with an iron deficiency, were recruited in this study. Subjects reported to have gastrointestinal disorders or to be in need of iron-based nutritional therapy. Recruitment was carried out through a routine visit when iron deficiency or very low daily iron administration was detected.

### Placement

General data were collected during recruitment: sex, age, voluntary habits, BMI, frequency of defecation, eating habits. In clinical practice iron supplementation has been suggested in anaemic patients or in patients with clinical or biochemical signs of iron deficiency.

Subjects were identified in group 1 or 2 for treatment A (Sucrosomial® Iron) or treatment B (Iron (II) sulfate). A 6 ml blood sample will be collected prior to administration of the iron-based products and initiation of treatment.

After 14 days, a blood sample will be collected for the determination of secondary results, and the protocol will be repeated with secondary administration of iron.

### Treatment

Recruited subjects are iron deficient and they will be treated with Sucrosomial® iron 30 mg or iron (II) sulfate 105 mg. The study was designed with open label and visible, non-randomised, with all subjects receiving both the Sucrosomial® iron composition and the iron (II) sulfate composition (open labeled not-randomized entry-controlled crossover design).

### Treatment Groups:

Group 1: i) 2 capsules/day of Sucrosomial® iron for 14 days; ii) 1 capsule/day iron (II) sulfate for 14 days.
Group 2: i) 1 capsule/day of iron (II) sulfate for 14 days; ii) 2 capsules of Sucrosomial® Iron for 14 days.

Between the two treatments (treatment A, Sucrosomial® iron; treatment B, iron (II) sulfate), all subjects will be subjected to a 4-day wash out period. Subjects will be moved from one treatment to another on an informed and open label basis.

The treatment lasts:
2 weeks of treatment with iron (II) sulfate or Sucrosomial® iron
4 day wash out
2 weeks of treatment with iron (II) sulfate or Sucrosomial® iron
   Faecal samples for microbiota analysis are collected at:
T0=before the initiation of treatment
T1=14 days
T2=18 days
T3=32 days
   and sent within 2 hours of collection to the laboratory where they are stored at a temperature of −80° C. to maintain DNA and RNA quality intact. Genomic DNA will be extracted from the faecal samples using QIAmp PowerFecal Pro DNA Kit (QIAGEN, Hilden, Germany). DNA concentration will be calculated by measuring OD260 nm and the DNA purity will be estimated by determining the OD260/OD280 and OD260/OD230 ratios using BioPhotometer D30 spectrophotometer (Eppendorf).

### Metagenomic Analysis

The DNA genome, extracted from the faecal samples, will be subjected to metagenomic analysis by sequencing the gene which encodes 165 rRNA bacteria. Subsequently, NGS (Next Generation Sequencing) and data analysis is carried out using Novogene. Different regions i.e. V3-V4 of the gene will be amplified using Phusion® High-Fidelity PCR Master Mix (New England BioLabs, USA). PCR products will be purified with QIAGEN Gel Extraction Kit (QIAGEN) and libraries generated with NEBNext® Ultra™ DNA Library Prep Kit for illumina and quantified with Qubit and qPCR. The sequencing amplicon will be carried out with the HiSeq illumina platform. The sequences obtained will be grouped into OUTs by 97% similarity of DNA sequence which is considered to collect homologous species. Sequence analysis will be carried out using the Uparse software. Phylogenetic relationships among all OUTs will be determined using the multiple comparison program MUSCLE. Alpha and beta diversity will be calculated from the results.

### Inclusion Criteria

Subjects from 18-70 years
Iron deficiency: low levels of iron in the blood, ferritin serum levels lower than 25 μg/l
BMI 18.5-24.9 kg/m2
iron deficiency symptoms include: extreme tiredness, weakness, pale skin, high heart rate or shortness of breath or laboured breathing, headache, inflammation or redness of the tongue
inadequate administration of iron through diet, previously evaluated by a nutritionist, normal calprotectin levels
written informed consent

### Exclusion Criteria

Any subject meeting at least one of the following criteria will be considered ineligible
a subject taking drugs that modify the microbiota composition such as PPIs (proton pump inhibitors), antibiotics, symbiotics or prolonged use of probiotics, in the 4 weeks prior to the initiation of the study
Subject diagnosed with IBD, chronic hepatitis or pancreatitis, recent infection with C. difficile, Salmonella, Campylobacter, Shigella or aggressive infections with E. coli
Subjects who have had a blood transfusion over the last 4 months
Pregnant subjects
Breastfeeding subjects up to 6 months prior to the initiation of the study
Smoking subjects who smoke more than one cigarette per week

### Data Collection

Faecal calprotectin. Gut microbiota is mainly dominated by the following 4 phyla: Firmicutes, Bacteroidetes, Actinobacteria, Proteobacteria.

### Statistical Analysis

A p-value lower than 0.05 is considered statistically significant. Analyses will be conducted using SPSS (version 21, IBM Corp, Armonk, NY).

Preferred embodiments FRPn of the present invention are reported below.
FRP1. An iron (III) formulation for use in a method for the treatment of a gastrointestinal inflammation in a subject who had been previously subjected to a therapeutic treatment of an iron deficiency by administering an iron (II) compound or salt to said subject,
   wherein said iron (III) formulation reduces the amount of pathogenic bacteria belonging to the phylum Proteobacteria present in the gut microbiota of the treated subject, and
   wherein said iron (III) formulation comprises or, alternatively, consists of:
(a) an iron (III) salt or complex,
(b) at least one phospholipid,
(c) a sucrester or sucrose esters.
FRP2. The iron (III) formulation for use according to FRP1, wherein said treated subject is a subject with a gastrointestinal inflammation and is also anaemic, who had been previously treated with a treatment based on an iron (II) compound or salt that was unsuccessful.
FRP3. The iron (III) formulation for use according to FRP1 or FRP2, wherein said treatment method is for treating a subject with gastrointestinal inflammation and anaemia, preferably said gastrointestinal inflammation being a relapsed form.
FRP4. The formulation for use according to any one of FRP1-3, wherein said treatment method is for reducing said gastrointestinal inflammation; preferably said inflammation is due to an infection.
FRP5. The formulation for use according to any one of FRP1-4, wherein said pathogenic bacteria belonging to the phylum Proteobacteria are selected from the group comprising or, alternatively, consisting of: Escherichia coli, Helicobacter pylori, Salmonella typhi, Vibrio cholerae Streptococchi.
FRP6. The formulation for use according to any one of FRP1-5, wherein said iron (Ill) formulation increases or modifies the variety of the gut microbiota of the treated subject.
FRP7. The formulation for use according to any one of FRP1-6, wherein said iron (Ill) formulation further comprises (d) a gelatinised or pre-gelatinised starch of plant origin; preferably rice starch or corn starch; more preferably pre-gelatinised rice starch.
FRP8. The formulation for use according to any one of FRP1-7, wherein said (b) at least one phospholipid consists of a lecithin; preferably a lecithin (E322) selected from the group consisting of: sunflower lecithin, corn lecithin, soy lecithin and mixtures thereof.
FRP9. The formulation for use according to any one of FRP1-8, wherein said (c) a sucrester or sucrose esters consists of sucrester E473; preferably a sucrester (e.g. E473) comprising from 30% to 95% by weight, preferably from 50% to 85% by weight, with respect to the total weight of the sucrester, at least one sucrose monoester with a fatty acid of plant origin, preferably wherein said fatty acid consists of stearic acid and/or palmitic acid.
FRP10. The formulation for use according to any one of FRP1-9, wherein said iron (Ill) formulation comprises or, alternatively, consists of:
(a) iron (Ill) pyrophosphate,
(b) sunflower lecithin;
(c) sucrester or sucrose esters; and
(d) pre-gelatinised rice starch;
preferably, wherein a [(c):(b)] by weight ratio between said (c) sucrester or sucrose esters and said (b) phospholipid or lecithin is comprised from 50:1 to 10:1, preferably from 40:1 to 10:1, more preferably from 30:1 to 15:1.
FRP11. The formulation for use according to any one of FRP1-10,
wherein said gastrointestinal infection is selected from the group consisting of:
intestinal inflammations, gastrointestinal ulcers, chronic gastritis, gastric atrophy, intestinal diverticula, diarrhoea, vomiting, gastrointestinal pain, nausea, constipation.
FRP12. The formulation for use according to any one of FRP1-11, wherein said subject is a subject having or at risk of a gastrointestinal infection;
preferably wherein said subject having or at risk of a gastrointestinal infection is selected from the group consisting of: subject with inadequate nutritional intake, subject living in inadequate hygiene conditions, celiac subject, subject with IBD, subject subjected to bariatric surgery, subject with gastritis, subject with atrophic gastritis, subject with gastroesophageal reflux disease (GERD).
FRP13. A composition for use according to any one of FRP1-12, wherein said composition comprises or, alternatively, consists of:
the formulation according to any one of the preceding claims, and
at least one pharmaceutical or food grade additive and/or excipient.

1. A method for the treatment of a gastrointestinal inflammation in a subject who had been previously subjected to a therapeutic treatment of an iron deficiency by administering an iron (II) compound or salt to said subject, comprising administering an iron (III) formulation to the subject, wherein said iron (III) formulation comprises or, alternatively, consists of:
(a) an iron (III) salt or complex,
(b) at least one phospholipid,
(c) a sucrester or sucrose esters
and wherein said iron (III) formulation reduces the amount of pathogenic bacteria belonging to the phylum Proteobacteria present in the gut microbiota of the treated subject.

2. The method according to claim 1, wherein said treated subject is a subject with a gastrointestinal inflammation and is also anaemic, who had been previously treated with a treatment based on an iron (II) compound or salt that was unsuccessful.

3. The method according to claim 1, wherein said subject has a gastrointestinal inflammation and anaemia, preferably said gastrointestinal inflammation being a relapsed form.

4. The method according to claim 1, wherein said administering reduces said gastrointestinal inflammation; preferably said inflammation is due to an infection.

5. The method according to claim 1, wherein said pathogenic bacteria belonging to the phylum Proteobacteria are selected from the group comprising or, alternatively, consisting of: Escherichia coli, Helicobacter pylori, Salmonella typhi, Vibrio cholerae Streptococchi.

6. The method according to claim 1, wherein said iron (III) formulation increases or modifies the variety of the gut microbiota of the treated subject.

7. The method according to claim 1, wherein said iron (III) formulation further comprises (d) a gelatinised or pre-gelatinised starch of plant origin; preferably rice starch or corn starch; more preferably pre-gelatinised rice starch.

8. The method according to claim 1, wherein said (b) at least one phospholipid consists of a lecithin; preferably a lecithin (E322) selected from the group consisting of: sunflower lecithin, corn lecithin, soy lecithin and mixtures thereof.

9. The method according to claim 1, wherein said (c) a sucrester or sucrose esters consists of sucrester E473; preferably a sucrester (e.g. E473) comprising from 30% to 95% by weight, preferably from 50% to 85% by weight, with respect to the total weight of the sucrester, at least one sucrose monoester with a fatty acid of plant origin, preferably wherein said fatty acid consists of stearic acid and/or palmitic acid.

10. The method according to claim 1, wherein said iron (III) formulation comprises or, alternatively, consists of:
(a) iron (III) pyrophosphate,
(b) sunflower lecithin;
(c) sucrester or sucrose esters; and
(d) pre-gelatinised rice starch;
preferably, wherein a [(c):(b)] by weight ratio between said (c) sucrester or sucrose esters and said (b) phospholipid or lecithin is comprised from 50:1 to 10:1, preferably from 40:1 to 10:1, more preferably from 30:1 to 15:1.

11. The method according to claim 1,
wherein said gastrointestinal infection is selected from the group consisting of:
intestinal inflammations, gastrointestinal ulcers, chronic gastritis, gastric atrophy, intestinal diverticula, diarrhoea, vomiting, gastrointestinal pain, nausea, constipation.

12. The method according to claim 1, wherein said subject is a subject having or at risk of a gastrointestinal infection;
preferably wherein said subject having or at risk of a gastrointestinal infection is selected from the group consisting of: subject with inadequate nutritional intake, subject living in inadequate hygiene conditions, celiac subject, subject with IBD, subject subjected to bariatric surgery, subject with gastritis, subject with atrophic gastritis, subject with gastroesophageal reflux disease (GERD).

13. A method according to claim 1, wherein said composition comprises or, alternatively, consists of:
the formulation according to any one of the preceding claims, and
at least one pharmaceutical or food grade additive and/or excipient.

14. An iron (III) formulation comprising:
(a) an iron (III) salt or complex,
(b) at least one phospholipid,
(c) a sucrester or sucrose esters.

15. The formulation of claim 14, wherein the formulation further comprises (d) a gelatinised or pre-gelatinised starch of plant origin; preferably rice starch or corn starch; more preferably pre-gelatinised rice starch.

16. The formulation of claim 14, wherein the said (b) at least one phospholipid consists of a lecithin; preferably a lecithin (E322) selected from the group consisting of: sunflower lecithin, corn lecithin, soy lecithin and mixtures thereof.

17. The formulation of claim 14, wherein said (c) a sucrester or sucrose esters consists of sucrester E473; preferably a sucrester (e.g. E473) comprising from 30% to 95% by weight, preferably from 50% to 85% by weight, with respect to the total weight of the sucrester, at least one sucrose monoester with a fatty acid of plant origin, preferably wherein said fatty acid consists of stearic acid and/or palmitic acid.

18. The formulation of claim 14, wherein the formulation comprises:
(a) iron (III) pyrophosphate,
(b) sunflower lecithin;
(c) sucrester or sucrose esters; and
(d) pre-gelatinised rice starch;
wherein a [(c):(b)] by weight ratio between said (c) sucrester or sucrose esters and said (b) phospholipid or lecithin is comprised from 50:1 to 10:1, preferably from 40:1 to 10:1, more preferably from 30:1 to 15:1.
